(19) United States
(12) Patent Application Publication
(10) Pub. No.: US 2021/0232567 A1
(43) Pub. Date: Jul. 29, 2021

(54) ANALYZER, ANALYSIS SYSTEM, AND ANALYSIS METHOD

(71) Applicant: toor Inc., Fukushima (JP)

(72) Inventors: Yoshio TAKAEDA, Fukushima (JP); Tetsuya KANADA, Fukushima (JP)

(21) Appl. No.: 16/333,816

(22) PCT Filed: Oct. 10, 2018
(86) PCT No.: PCT/JP2018/037708

§ 371 Date: Mar. 15, 2019

(51) Int. Cl. G06F 16/23; G07C 5/00; G07C 5/08; G16H 50/30; G10L 25/63; G06K 9/00

(52) U.S. Cl. CPC G06F 16/2379; G07C 5/008; G07C 5/08; G06Q 50/26; G10L 25/63; G06K 9/00624; G16H 50/30

(57) ABSTRACT

An analyzer includes: a reference map receiver configured to receive information regarding a reference map in which reference data are plotted in advance on the basis of similarity index between the reference data; and a new data plotting unit configured to plot new data different from the reference data on the reference map on the basis of the similarity index between the new data and a part or all of the reference data on the reference map.

### BACKGROUND

### 1. Field of the Disclosure

This disclosure relates to an analyzer, an analysis system, and an analysis method.

### 2. Discussion of the Background Art

An analysis system that analyzes data created at each site has been proposed (for example, see Patent Document 1). In the analysis system of Patent Document 1, terminal devices are disposed in each site, and a control center analyzes data from each terminal device.

In the analysis system of Patent Document 1, since the control center analyzes the data from each terminal device, disadvantageously, it is difficult to perform the analysis when the terminal device and the control center are not connected to each other via a communication network. In addition, there is a demand for the analysis result in each site in some cases. Furthermore, it is desirable that a computation processing load of each site is small.

### CITATION LIST

### Patent Documents

Patent Document 1: Japanese Unexamined Patent Application Publication No. 09-251591

### SUMMARY

In this regard, an object of this disclosure is to enable each site to analyze data created in each site with a small computation processing load.

According to an aspect of this disclosure, there is provided an analyzer including: a reference map receiver configured to receive a reference map in which reference data are plotted in advance on a map on the basis of similarity index between the reference data; and a new data plotting unit configured to plot new data different from the reference data on the reference map on the basis of similarity index between the new data and all or a part of the reference data on the reference map.

According to another aspect of this disclosure, there is provided an analysis system including: the analyzer according to this disclosure; and a reference map creating device provided with a reference map creating unit that obtains data used as the reference data and creates the reference map on the basis of similarity index between the reference data and configured to provide the reference map to the analyzer.

According to further another aspect of this disclosure, there is provided an analysis method, using an analyzer, the analysis method including: receiving a reference map created in advance on the basis of similarity index between reference data; and plotting new data different from the reference data on the reference map on the basis of similarity index between the new data and all or a part of the reference data on the reference map.

According to this disclosure, it is possible to enable each site to analyze data created in each site with a small computation processing load.

### BRIEF DESCRIPTION OF THE DRAWINGS

FIG. 1 illustrates an exemplary configuration of an analysis system according to a first embodiment;

FIG. 2 illustrates an exemplary area on a reference map;

FIG. 3 illustrates an exemplary configuration of an analysis system according to a second embodiment;

FIG. 4 illustrates exemplary new data belonging to none of the areas;

FIG. 5 illustrates an exemplary area whose area attribute changes;

FIG. 6 illustrates an exemplary configuration of an analysis system according to a fourth embodiment;

FIG. 7 illustrates an exemplary presentation;

FIG. 8 illustrates an exemplary presentation according to a fifth embodiment;

FIG. 9 illustrates an exemplary configuration of an analysis system according to a sixth embodiment;

FIG. 10 illustrates an exemplary configuration of an analysis system according to a seventh embodiment;

FIG. 11 illustrates an exemplary system configuration according to an eighth embodiment; and

FIG. 12 illustrates an exemplary method of plotting new data.

### DETAILED DESCRIPTION OF THE PREFERRED EMBODIMENT

Embodiments of this disclosure will now be described in details with reference to the accompanying drawings. Note that this disclosure is not limited to the following embodiments. Such embodiments are merely for exemplary purposes, and various modifications or changes may be possible on the basis of knowledge of those skilled in the art. Note that like reference numerals denote like elements throughout this specification and the attached drawings.

### First Embodiment

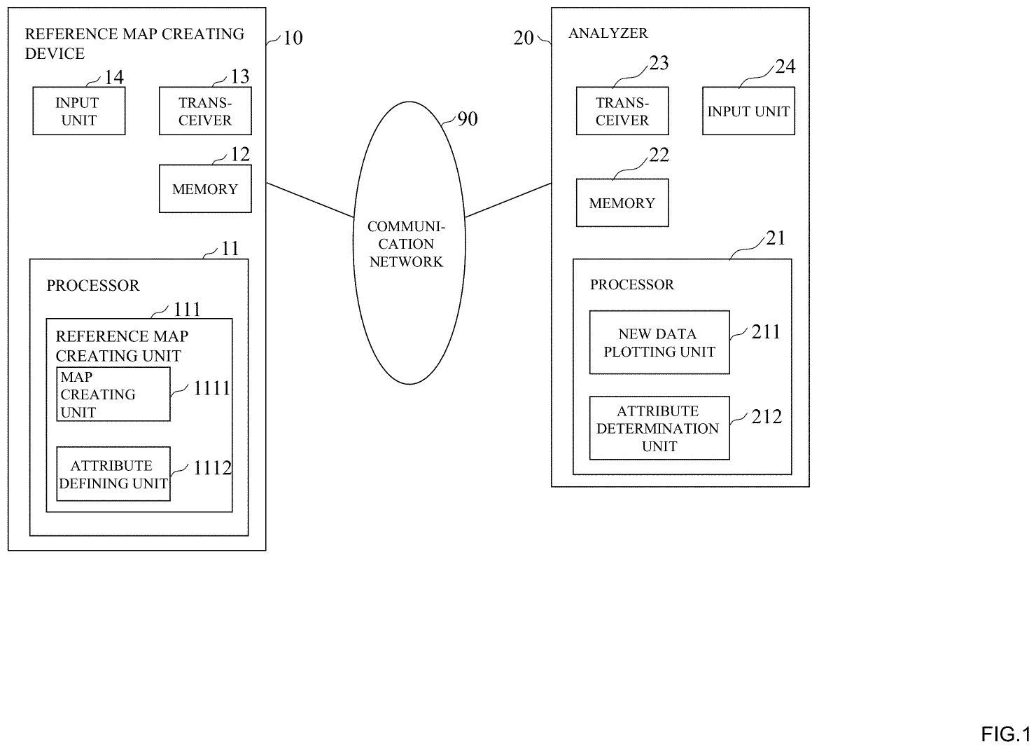

FIG. 1 illustrates an exemplary configuration of an analysis system according to this disclosure. The analysis system according to this disclosure has a reference map creating device 10 and an analyzer 20 connected to each other via a communication network 90. In FIG. 1, the reference map creating device 10 and the analyzer 20 are connected to each other using a one-to-one connection scheme by way of example. However, without limiting thereto, any scheme such as one-to-many or many-to-many scheme may also be employed. If a plurality of analyzers 20 are employed, each analyzer 20 is disposed in different sites.

The reference map creating device 10 provides a reference map to the analyzer 20. The analyzer 20 determines an attribute of new data using the reference map provided from the reference map creating device 10 when new data different from the reference map is obtained.

The reference map creating device 10 may include any computer having a processor 11, a memory 12, a transceiver 13, and an input unit 14. The processor 11 of the reference map creating device 10 has a reference map creating unit 111. The reference map creating unit 111 has a map creating unit 1111 and an attribute defining unit 1112.

The analyzer 20 has a processor 21, a memory 22, a transceiver 23, and an input unit 24. The processor 21 of the analyzer 20 has a new data plotting unit 211 and an attribute determination unit 212. The transceiver 23 functions as a reference map receiver to execute a step of receiving a reference map. The new data plotting unit 211 executes a step of plotting new data on a reference map.

The map creating unit 1111 obtains data used as reference data and creates a map. The map is a map in which reference data are plotted on the basis of similarity index between the reference data. For example, the map creating unit 1111 converts each reference data into vector data and obtains a plot location of each reference data using a distance between the vector data. As a result, a map is created, in which points representing each reference data are plotted on the basis of the similarity index between the data. Here, the distance between the vector data may be an Euclidean distance or an inner product space distance, or may be obtained using any computation method such as an outer product method.

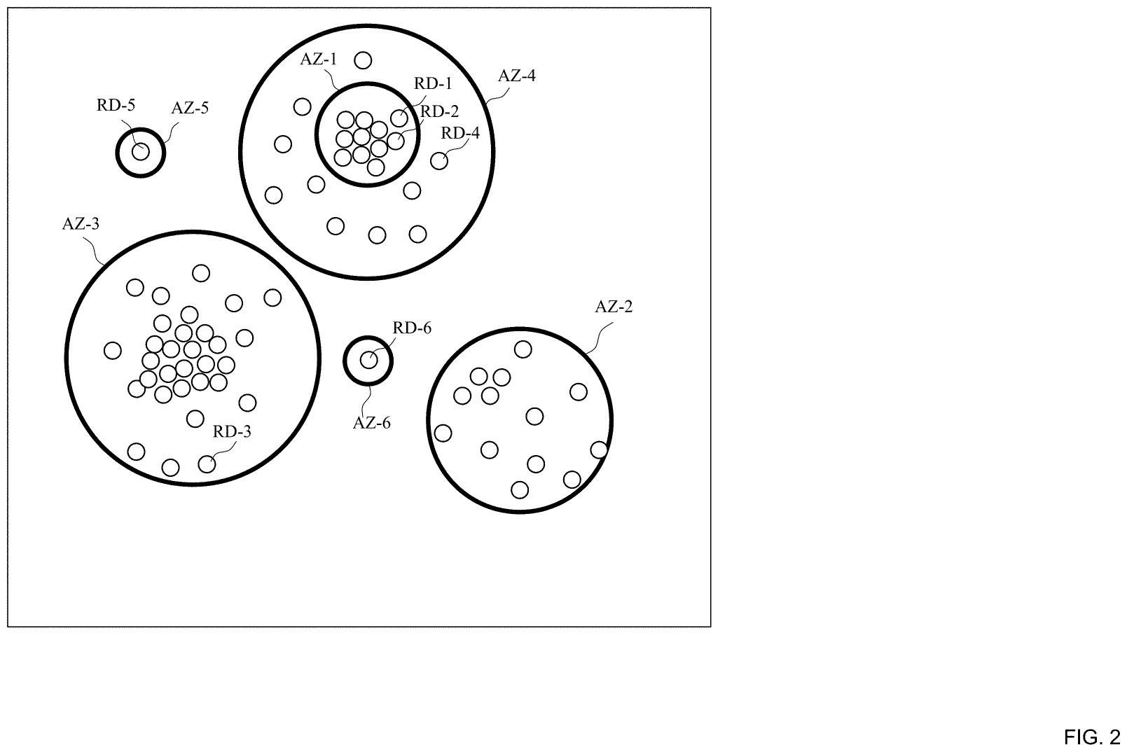

FIG. 2 illustrates an exemplary map. Data having high similarity are arranged close to each other, and data having low similarity are arranged far from each other. Preferably, the data arranged close to each other are arranged in a more accurate distance. The reference data on the map may be arranged on a plane as illustrated in FIG. 2. Alternatively, the reference data on the map may be arranged in a vector space having an arbitrary dimension such as a spherical surface.

The attribute defining unit 1112 defines a single area or a plurality of areas on the reference map and defines attributes for each area depending on characteristics of the reference data included in each area.

The transceiver 13 transmits the reference map to the analyzer 20, and the transceiver 23 receives the reference map from the reference map creating device 10. The reference map is stored in the memory 22. The reference map contains reference data, area information, and area attributes. The area information contains information regarding the reference data included in the area and information regarding the location and range of the area on the map. The reference map may also include attribute data of each reference data such as time at which the data are created, data types, and identification information of the device which creates the data.

For example, in the case of the reference map of FIG. 2, the reference map contains identification information of the reference data RD-3, identification information of the area AZ-3, and the area attribute of the area AZ-3 associated with each other.

Here, two or more areas and two or more area attributes may be defined for a single location on the reference map. For example, the identification information of the reference data RD-1 may be associated with the areas AZ-1 and AZ-4.

In some cases, the number of the reference data is small as in the reference data RD-5 and RD-6. In a case where a distribution range of such a common area attribute is not clear, locations of the reference data RD-5 and RD-6 and a certain range from such locations are defined as the area.

The new data plotting unit 211 plots new data on the reference map as new data different from the reference data is obtained. For example, in a case where the similarity index of the data is a distance between the vector data, the new data plotting unit 211 converts the new data into vector data, calculates a distance between the vector data of the new data and the vector data of each reference data, and extracts the reference data having a distance close to the vector data. In this case, the new data plotting unit 211 may extract all the reference data. In addition, the new data plotting unit 211 plots the new data on the reference map on the basis of the distance between the extracted reference data and this new data. A specific plotting method will be described below.

The attribute determination unit 212 determines the plotted area of the new data and determines the area attribute of this area as an attribute of the new data. For example, in a case where the new data is arranged inside of the area AZ-3, the attribute determination unit 212 determines that the new data has an area attribute of the area AZ-3.

In this manner, the analyzer 20 can extracts the area attribute of the new data merely by specifying the plot location of the new data on the reference map. For this reason, in the analysis system according to this embodiment, the computation processing of the analyzer 20 is small. Therefore, it is possible to perform the analysis of the analyzer 20 at a high speed.

Here, the analysis in the analyzer 20 is preferably performed stably at a high speed. For this reason, the new data plotting unit 211 and the attribute determination unit 212 perform the processing using hardware such as a field programmable gate array (FPGA) in some cases.

As described above, according to this embodiment, the analyzers 20 are distributed in the sites other than the reference map creating device 10, and each analyzer 20 determines the area attribute of the new data. Therefore, the analysis of the data created in each site can be performed in each site. In addition, the plot location of the new data on the reference map can be computed with a small computation load, compared to creation of the reference map itself. Therefore, it is possible to reduce the computation processing load of each analyzer 20.

In many cases, generally, the analyzer 20 is preferably arranged close to a data creation place as the data amount to be analyzed increases. In addition, the analyzer 20 is preferably arranged far from the data creation place as the data amount to be analyzed decreases. However, this is not necessarily the case, but the analyzer 20 may be arranged flexibly depending on the use purpose or the like.

Although new data is plotted on the reference map on the basis of similarity index with all the reference data in this embodiment, this disclosure is not limited thereto. The new data may be plotted on the reference map on the basis of the similarity index with a part of the reference data. For example, the reference data used to calculate the similarity index with the new data may be set as one for each area, and calculation of the similarity index between the new data and other reference data belonging to the common area is omitted. In addition, out of the areas, an area having a low similarity is excluded, and similarity index between the new data and each reference data included in the area having a high similarity are calculated, so that the plot location of the new data is obtained. As a result, it is possible to further reduce a computation processing load of the analyzer 20.

### Second Embodiment

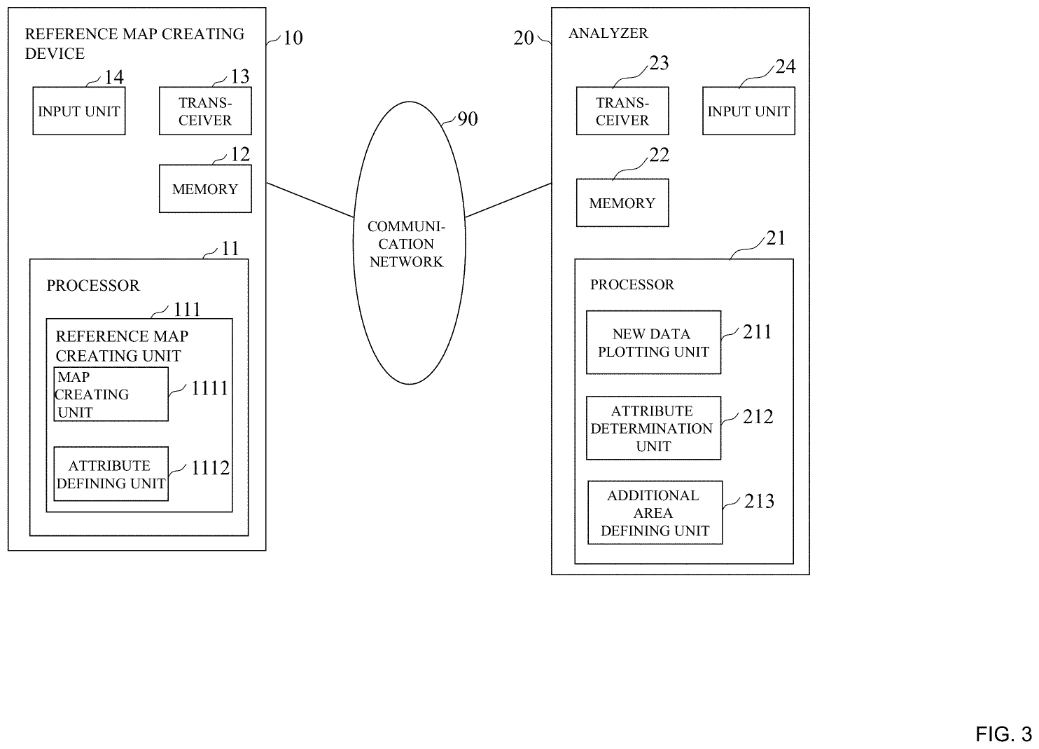

FIG. 3 illustrates an exemplary configuration of the analysis system according to this disclosure. In the analysis system according to this disclosure, the processor 21 of the analyzer 20 further has an additional area defining unit 213.

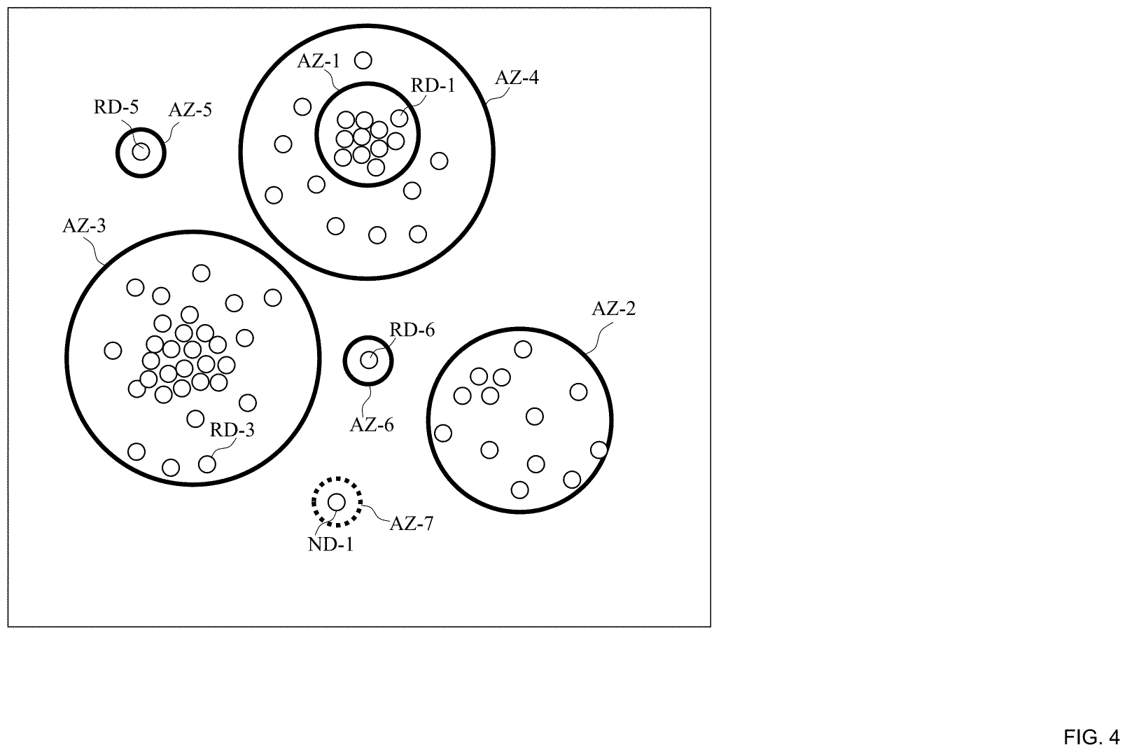

In some cases, the area attribute may not be defined for the area where the new data is plotted. For example, as in the new data ND-1 of FIG. 4, the area attribute is not defined for the plot location in some cases. In this case, the attribute determination unit 212 outputs the new data ND-1 and information regarding the plot location to the additional area defining unit 213 along with the reference map.

The additional area defining unit 213 compares the attribute of the new data ND-1 with each area attribute and determines the attribute of the plot location of the new data ND-1. In addition, the additional area defining unit 213 defines the attribute of the plot location of the new data ND-1 in the area attribute having a certain range from the new data ND-1.

The transceiver 23 transmits all of the new data and additional area information to the reference map creating device 10. The additional area information includes information regarding the new data ND-1, a location of the new data ND-1, and an area attribute within a certain range from the new data ND-1. The reference map creating device 10 stores the information received from the analyzer 20 in the memory 12. The reference map creating unit 111 uses the information stored in the memory 12 in addition to all or a part of the data on the initial reference map when the reference map is updated.

The reference map creating device 10 creates a new reference map in which the new data ND-1 is included in the reference data. In this case, the area AZ-7 is newly defined. The reference map creating device 10 transmits the new reference map to the analyzer 20. As a result, each analyzer 20 can perform the analysis using the new reference map in which the area AZ-7 is defined.

The reference map creating device 10 may transmit the new reference map to each analyzer 20 at an arbitrary timing. For example, the new reference map may be transmitted when the new reference map is created, or may be transmitted on a periodic basis.

As described above, according to this embodiment, the reference map is updated using the new data. For this reason, according to this embodiment, it is possible to automatically cope with a newly created area attribute as well.

### Third Embodiment

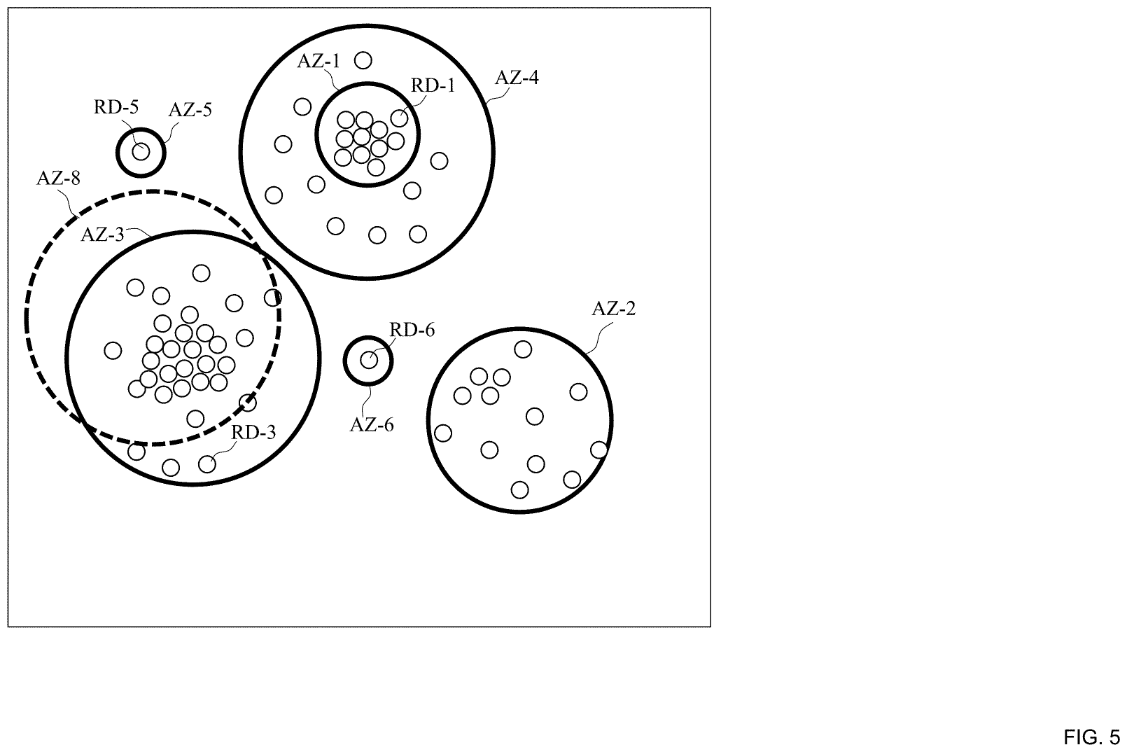

Some data slowly changes depending on various conditions such as time and environment. For example, a change in the average temperature depending on the season may have an influence in some cases. In the case of such data, as illustrated in FIG. 5, the area AZ-3 having an area attribute indicating a normal state moves to the area AZ-8 with a lapse of time, so that the reference data RD-3 belonging to the area AZ-3 may not be in the normal state.

In the system according to this disclosure illustrated in FIG. 1, it is not desirable to use the area attribute of the area AZ-3 when the attribute determination unit 212 determines the area attribute. In this regard, according to this embodiment, the reference map is updated depending on a change of the area attribute.

The analyzer 20 periodically transmits the new data accumulated in the memory 22 to the reference map creating device 10. As the transceiver 13 of the reference map creating device 10 receives the new data from each analyzer 20, the new data is stored in the memory 12.

The map creating unit 1111 reads the new data stored in the memory 12 as reference data and creates the reference map using the reference data. The attribute defining unit 1112 defines the area attribute for each area on the reference map using the attribute of the reference data distributed in the area. As a result, it is possible to create a new reference map by reflecting a change of the area attribute.

Here, the map creating unit 1111 preferably reads data including the new data in the reference data within a certain period of time from the current time as the reference data. In this case, the existing reference data included in the new reference map may contain all or a part of the existing reference data. For example, in order to prevent an increase of the computation processing load of the analyzer 20, the number of data included in the new reference map is preferably set to be constant.

The transceiver 13 transmits the new reference map to the analyzer 20. Each analyzer 20 stores the new reference map in the memory 22 as the new reference map is received from the reference map creating device 10. The subsequent operation is similar to that of the embodiment described above.

As described above, according to this embodiment, the reference map is updated using the new data. For this reason, according to this embodiment, it is possible to automatically cope with the data having an area attribute changing depending on various conditions such as time and environment as well.

### Fourth Embodiment

In this embodiment, an example will be described, in which one or more sensors are mounted on a device, reference data and new data are detected using the sensors as sensor data, and an area attribute indicates a state of the device where the sensors are mounted.

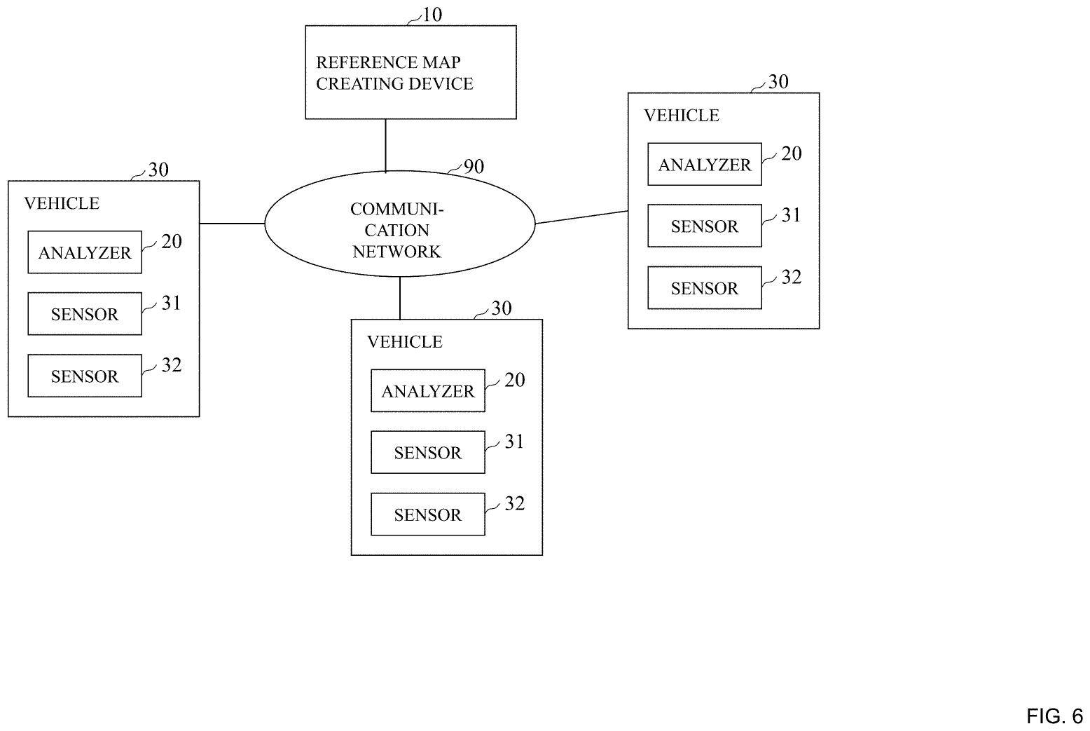

FIG. 6 illustrates an exemplary system configuration according to this embodiment. In the system according to this embodiment, sensors 31 and 32 and an analyzer 20 are mounted on a vehicle 30. Although FIG. 6 illustrates an example in which two types of sensors 31 and 32 are provided in the vehicle 30, only a single type of the sensor 31 or three or more types of sensors 31 may be mounted on the vehicle 30. The reference map creating device 10 is managed by a manufacturer or the like of the vehicle 30 that maintains normal values of the sensors 31 and 32 in advance. The configurations of the reference map creating device 10 and the analyzer 20 are similar to those illustrated in FIGS. 1 and 3.

The reference map creating device 10 transmits the reference map to each vehicle 30. The reference map is updated appropriately. The sensors 31 and 32 are arbitrary detection devices for analyzing a state of the vehicle 30, whether the vehicle 30 is normal or abnormal, a type of a failure or abnormality state, and whether or not the components where the sensors 31 and 32 are mounted are normal or abnormal. An analysis result is a content that a driver wants to know in real time. In this regard, according to this embodiment, the analyzer 20 analyzes the state in real time.

The vehicle 30 is an arbitrary car including an automobile, a motor-equipped bicycle, a light vehicle, a bus, and a railway car. The sensors 31 and 32 are arbitrary sensors mounted on the vehicle, such as a vehicle speed sensor, an acceleration sensor, a vehicle position sensor, a collision detection sensor, a rear monitoring camera, a rear obstacle sensor, a side obstacle sensor, an inter-vehicle distance sensor, a road surface sensor, a magnetic sensor, and a driver state sensor. The sensors 31 and 32 may be sensors that detect a state of any component of the vehicle 30, such as a steering angle sensor of a steering wheel, a fire detection sensor mounted near an engine, a tire air pressure sensor, and a wheel or engine vibration sensor.

The sensor data may be raw data output from the sensors 31 and 32, or may be processed data obtained by computing the data output from the sensors 31 and 32. The processed data includes, for example, an average value, a median value, a maximum value, a minimum value, a range, and a most frequent value of the sensor data from the sensor 31. In addition, the sensor data also includes a frequency spectrum of vibration or a spatial frequency spectrum of an image.

The reference map creating device 10 collects the sensor data of the sensors 31 and 32 provided in each vehicle 30 and creates a reference map for each vehicle 30. According to this embodiment, the reference data are distributed on the basis of the values of each sensor 31 and 32. For this reason, the area attribute according to this embodiment includes a state of the vehicle 30, whether the vehicle 30 is normal or abnormal, a type of a failure or abnormality state, and whether or not components where the sensors 31 and 32 are mounted are normal or abnormal.

The reference map creating device 10 transmits the reference map to each vehicle 30 as the reference map is created. The analyzer 20 receives the reference map corresponding to the vehicle 30 where the analyzer 20 is mounted from the reference map creating device 10 and stores it in a memory (reference numeral 22 in FIG. 1).

When new sensor data is created in the sensor 31, the analyzer 20 stores the new sensor data in the memory (reference numeral 22 in FIG. 1) as new data. The analyzer 20 reads the reference map corresponding to the vehicle 30 where the analyzer 20 is mounted from the memory 22, plots the new data on the reference map, and determines the attribute of the new data on the basis of the plot location.

For example, in a case where the sensor 31 is an engine vibration sensor, the plot location of the new data is the area AZ-3, and the area attribute of the area AZ-3 indicates that the engine vibration has a normal value, the analyzer 20 determines that the engine vibration is within a normal range.

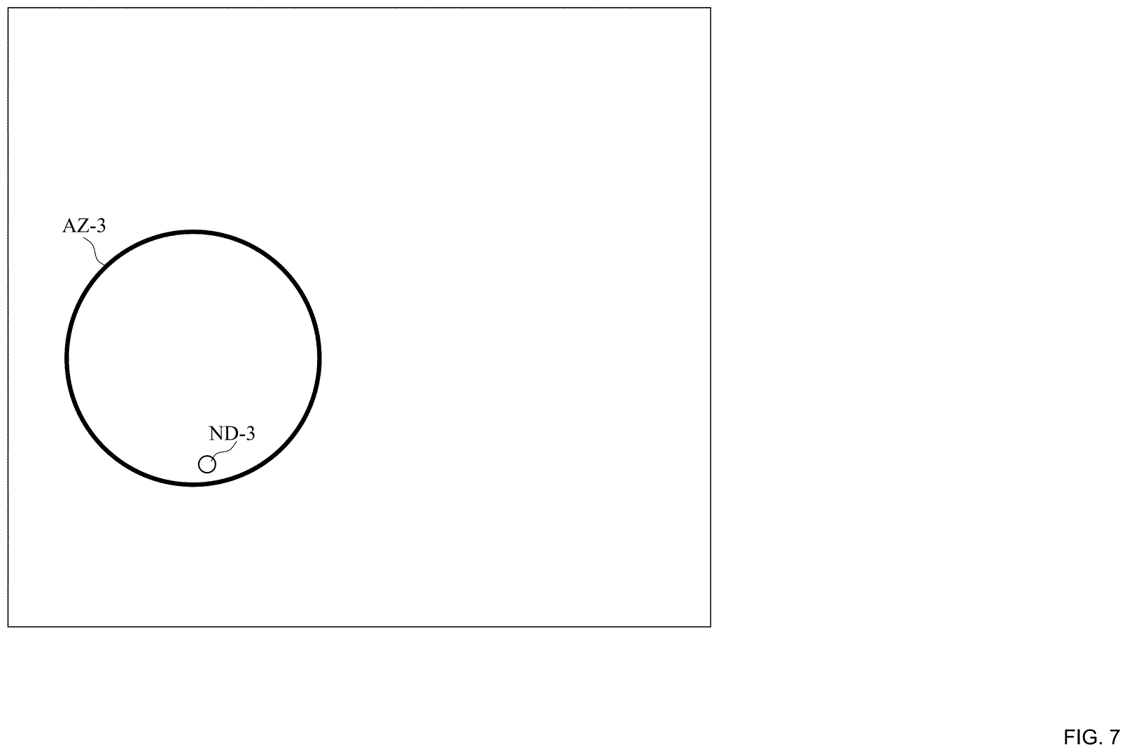

The analyzer 20 preferably displays the determination result on an arbitrary monitor provided in the vehicle 30. FIG. 7 illustrates an exemplary presentation. The area AZ-3 indicating a normal range and the new data ND-3 are displayed. The new data ND-3 is located in an end of the area AZ-3. For this reason, a user of the analyzer 20 can visually recognize that this is a value of the sensor 31 almost deviated from the normal range. As described above, according to this embodiment, it is possible to determine a state of the component of the vehicle 30, or whether the vehicle 30 has a normal or abnormal state. Note that the sensors 31 and 32 may be combined to determine the state, normality, or abnormality of the vehicle 30.

As described above, according to this disclosure, it is possible to determine whether or not the vehicle 30 is within a normal range, whether or not there is a possibility of abnormality, what kind of state the vehicle 30 is within the normal range, and what kind of abnormality is, for each vehicle 30 using the analyzer 20 in real time.

Note that the sensor data from the sensors 31 and 32 may have difference data characteristics depending on the installation states of the sensors 31 and 32 or the like. In this regard, the reference data are preferably collected while the sensors 31 and 32 are mounted on the vehicle 30. For example, the reference map may be prepared by a vehicle manufacturer or a machine manufacturer, or may be individually created while the sensors are actually mounted in a factory or the like.

Although a case where the sensor data from the sensors 31 and 32 are used has been described in this embodiment, log data output by a device itself may also be employed instead of the sensor data. In this case, vector data obtained by combining both the sensor data and the log data may also be employed as the reference data.

In this embodiment, the device where the sensor is mounted is a vehicle by way of example. However, the disclosed device is not limited thereto. This disclosure may also be applicable to an arbitrary device that preferably continuously checks a condition, instead of the vehicle 30. For example, the vehicle 30 according to this embodiment may include any device having a drive mechanism or a movable part such as an elevator, an escalator, a generator, a belt conveyor, an aircraft, or an industrial robot. For example, the arbitrary device may include a servo motor, an inverter, a speed reducer, a compressor, or the like. For such devices and components, arbitrary data output from devices and components, such as torque data, control currents, or voltage values may be employed instead of the sensor data. The aircraft includes an airship, a helicopter, or an airplane.

The sensor 31 according to this embodiment may include an arbitrary sensor mounted on a device. For example, the sensor 31 includes a flow sensor that detects a flow rate of a fluid in a pipe or a tank, a vibration sensor that detects vibration caused by a flow, or a current sensor that detects a current flowing through the circuit.

When a change occurs in a fluid or gas in a pipe, a vibration or temperature changes in the pipe. For this reason, the sensor 31 may be a sensor that detects a vibration, a flow rate, or a temperature generated in the pipe for flowing a fluid or gas. As a result, according to this disclosure, it is possible to detect any abnormality contributing to a flow of the fluid or gas in real time, regarding presence or absence of a variation in a vortex or flow of the fluid or gas flowing through the pipe, whether or not a driving unit for flowing the fluid or gas is normally operated, whether or not there is looseness in connection of the pipes, and the like.

If a change occurs in an electrical component, a current of the path connected to the electric component changes. For this reason, the sensor 31 may be a sensor that detects a current or voltage of a path connected to an electric component. As a result, according to this disclosure, it is also possible to determine, in real time, an electrical abnormality such as whether or not an abnormality occurs in operation of the electric component, or whether or not a connection failure occurs in connection of the electric component.

### Fifth Embodiment

In this embodiment, an example will be described, in which the sensor data described in the fourth embodiment includes vibration data, and the area attribute includes a deterioration state of a road surface on which the vehicle travels. According to this disclosure, a system configuration similar to that of the fourth embodiment may be employed.

Generally, at least any one of the sensors 31 and 32 is an arbitrary device capable of detecting vibration of a vehicle in a vertical direction (hereinafter, referred to as a z-axis direction). However, the sensors 31 and 32 may include any device capable of detecting vibration of a vehicle in a travel direction (hereinafter, referred to as an x-axis direction) or a direction perpendicular to the vertical direction and the travel direction (hereinafter, referred to as a y-axis direction). The vibration includes slow vibration or fast vibration, and is not limited by a frequency range. The vibration may be detected using an acceleration sensor capable of measuring an acceleration.

According to this embodiment, vibration data divided into predetermined segments are used as the reference data and the new data. The segment is an arbitrary region serving as a determination target of the deteriorated state, and includes, for example, a geographical section of a road or a railroad track, or a temporal section divided on a fixed time basis. According to this embodiment, attribute information by which the segment serving as a determination target of the deteriorated state can be specified is associated.

Assuming that “Ai(t)” denotes vibration data of a segment “i”, a vibration data change “A(t)” can be expressed as:

(Formula 1)

A(t)=ΣAi(t) (where “i”=1,2,3, . . . ,N−1, and N) (1).

Here, “N” denotes a total number of the segments.

The vibration data divided into segments are converted into frequency spectra and are sampled as discrete frequency values. As a result, vector data having an amplitude of each frequency component in each dimensional value are created as vibration data of each segment. This vector data is used as the reference data and the new data according to this embodiment. Here, the vibration detected by the sensor 31 or 32 is different depending on the type of the vehicle, the specification of the damper provided in the vehicle, or the like. Therefore, weighting or standardization may be applied to each dimension so that the difference between the vehicles of the vibration data is reduced.

The reference map creating device 10 collects the vibration data of the sensors 31 and 32 provided in each vehicle 30 and creates the reference map. The analyzer 20 receives the reference map from the reference map creating device 10 and stores it in the memory (reference numeral 22 in FIG. 1). The subsequent processings of each device are similar to those of the fourth embodiment.

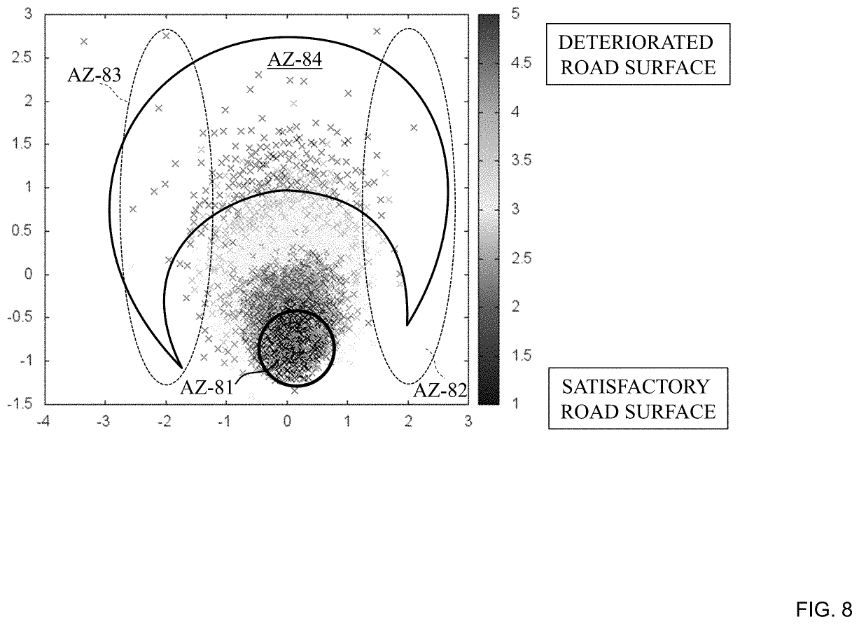

In the case of well-maintained roads or tracks, the plots are distributed in the area AZ-81 in FIG. 8. Here, each plot corresponds to each road segment one by one. In comparison, when the road is significantly deteriorated, the segment data are distributed in the area AZ-84 far from the area AZ-81. Even when the deterioration is significant, the area AZ-82 in which the segments having a lot of fine unevennesses are distributed is distributed apart from the area AZ-83 in which road segments having large undulations in a traveling direction are distributed.

The analyzer 20 reads the reference map from the memory 22, plots the new data on the reference map, and determines a deterioration state of the travel road surface on the basis of the plot location. For this reason, it is possible to determine a road surface state during the traveling of the vehicle 30 in real time.

As described above, according to this disclosure, it is possible to determine, in real time, what kind of deterioration state the traveling road surface of the vehicle 30 has in addition to the road deterioration level using the analyzer 20.

### Sixth Embodiment

In this embodiment, an example will be described, in which the reference data and the new data are biological data such as medical data, and the area attribute is a state of a living body such as a health state of a human being or animal, an illness type, or a degree of the illness.

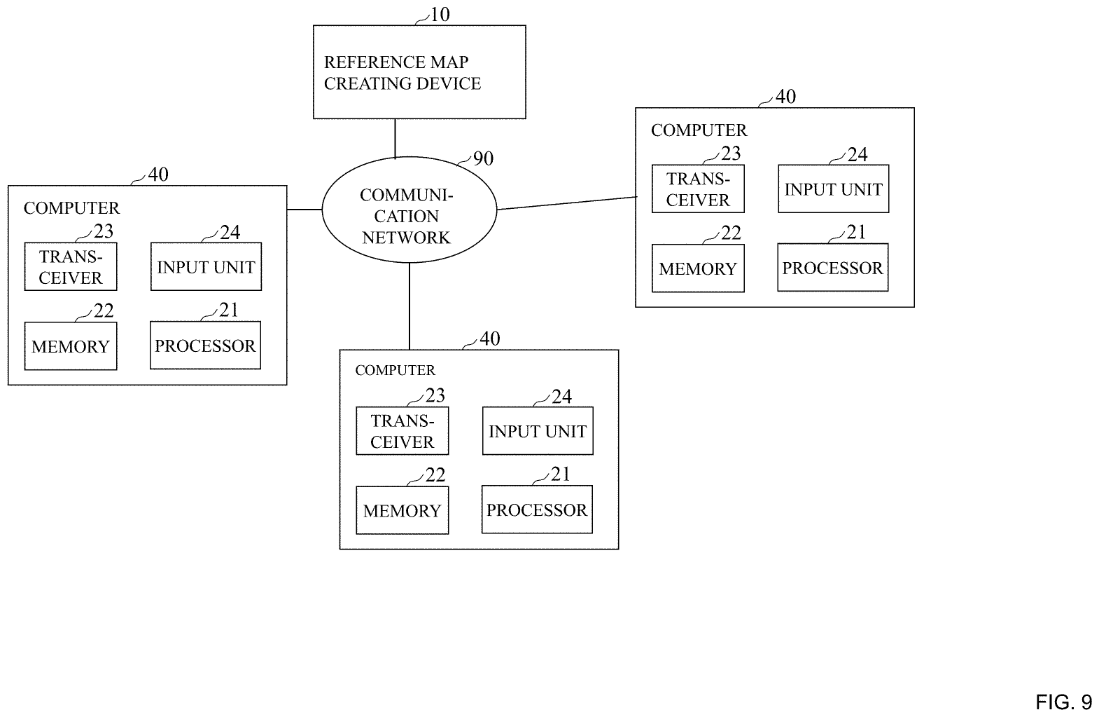

FIG. 9 illustrates an exemplary system configuration according to this embodiment. In the system according to this embodiment, a computer 40 functions as the analyzer 20 of FIG. 1. The computer 40 is a computer used by a doctor who is not a specialist of a private hospital or a rural hospital, or a computer used by an individual.

In a rural hospital, although an examination environment can be prepared, it is difficult to make a specialist resident. In addition, An environment in which patients suffering from diabetes or the like can conduct an examination by themselves is being provided. Meanwhile, a diagnosis result is the content the patient wants to know quickly. In this regard, according to this embodiment, a computer 40 disposed in a place where the biological data is detected performs the analysis by using the biological data obtained from an examination or the like as the reference data and the new data, and the analysis result is output to a monitor (not shown).

The biological data is examination data obtained by taking an examination target sample such as blood, exhalation, cerebrospinal fluid, urine, or a part of tissues and analyzing the sample. The biological data includes not only the data obtained from the examination target sample but also data obtained by performing a biological examination, a clinical examination including a physiological (function) examination, a radiation-related examination, and an endoscopic examination or interview data. The biological data includes a breathing sound, a heartbeat sound, electrocardiograms, or the like. The biological data includes an X-ray image, a magnetic resonance imaging (MRI) image, a computed tomography (CT) image, or the like.

In this regard, according to this embodiment, a specialized hospital or laboratory has the reference map creating device 10, creates a reference map from data of many examinees, and transmits the reference map to each computer 40. In addition, each computer 40 receives the reference map from the reference map creating device 10 and stores it in the memory 22. Each computer 40 uses the examination data obtained through the examination as new data and determines the area attribute on the reference map of the new data. As a result, according to this embodiment, at an arbitrary site where the computer 40 is disposed, such as a clinic or a rural hospital, it is possible to obtain the area attribute at a stage of obtaining the examination result using the examination data. That is, according to this embodiment, even when a specialist is absent, it is possible to perform a primary diagnosis for a patient. Here, in the case of a patient, the reference map may be commonly used in each hospital. However, the reference map may be different depending on age, gender, race, or the like.

The computer 40 inputs the examination data as new data using the input unit 24. The input unit 24 includes an arbitrary function capable of inputting data to the computer 40, such as a keyboard, a mouse, and a scanner. The computer 40 stores the new data in the memory 22. In addition, the computer 40 reads the reference map from the memory 22, plots the new data on the reference map, and determines the area attribute of the new data on the basis of the plot location.

For example, in a case where the new data is examination data of diabetes, the plot location of the new data is the area AZ-3 of FIG. 2, and the area attribute of the area AZ-3 is a normal value of the examination data, the analyzer 20 determines that the examination data is within a normal range. Similar to the fourth embodiment illustrated in FIG. 7, it is preferable that the area and the new data are displayed in this embodiment.

As described above, according to this disclosure, it is possible to determine and display, in real time, whether or not the examination data of the medical field is within a normal range, whether or not there is a possibility of abnormality, what kind of state the patient has within the normal range, what kind of abnormal state the patient has using the computer 40.

According to this disclosure, by accumulating the new data for each patient, it is possible to trace a patient's state as a part of clinical records across a long term. In addition, there may be a service of delivering a determination result of this embodiment as a primary diagnosis result along with the examination result such as examination data of clinical examination.

### Seventh Embodiment

In the seventh embodiment, an example will be described, in which reference data and new data are sound spectral data and biological data, and the area attribute includes a talker's psychological state.

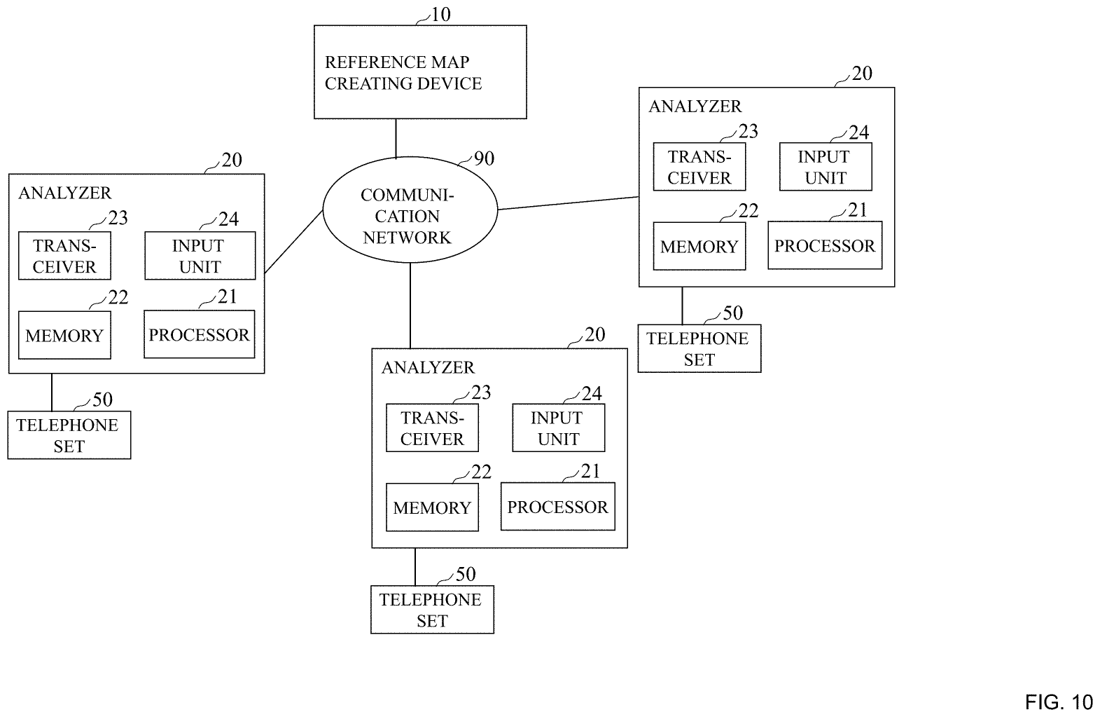

FIG. 10 illustrates an exemplary system configuration according to this embodiment. In the system according to this embodiment, each analyzer 20 is connected to each telephone set 50. The telephone set 50 is a part of many telephone sets provided in a call center.

A lot of calls are generated in the call center, and the contents are diverse. The map creating unit (reference numeral 1111 in FIG. 1) provided in the reference map creating device 10 separates sounds of many calls into segments of a certain time period, generates sound frequency spectral data for each segment, and creates a reference map using the sound frequency spectral data.

The sound tones vary depending on a talker's psychological state. For this reason, the talker's psychological state appears in the spectral data. For example, if the talker has a tense state, the sound spectral data has a high tone and a fast speed. If the talker has a depressed state, the sound spectral data has a low tone and a slow speed. If the talker has a calm state, the sound spectral data has a clear and stable tone. In this manner, the spectral data are distributed on the map depending on the talker's occasional psychological state. The attribute defining unit (reference numeral 1112 in FIGS. 1 and 3) according to this embodiment defines this talker's psychological state as the area attribute.

As the reference map is created, the reference map creating device 10 transmits the reference map to each analyzer 20. The analyzer 20 receives the reference map from the reference map creating device 10 and stores it in the memory (reference numeral 22 in FIG. 1).

When a call is placed on the telephone set 50 connected to the analyzer 20, the analyzer 20 acquires a sound from the telephone set 50 and converts it into spectral data. This spectral data becomes new data. The analyzer 20 plots the new data on the reference map and determines the area attribute of the new data on the basis of the plot position.

For example, if the plot location of the new data is the area AZ-3 in FIG. 2, and the area attribute of the area AZ-3 is a tens state, the analyzer 20 determines that the talker has a tense state.

The analyzer 20 preferably displays the determination result on a display common to the analyzer 20 or a floor of the call center. For example, if the talker has a calm state, a blue color is displayed. If the talker has an irritated state, an orange color is displayed. If the talker is in anger, a red color is displayed.

As described above, according to this disclosure, it is possible to allow the call center to check the talker's psychological state at each telephone set 50 in real time.

### Eighth Embodiment

In this embodiment, an example will be described, in which the reference data and the new data are image data, and the area attribute is identification information of the image data.

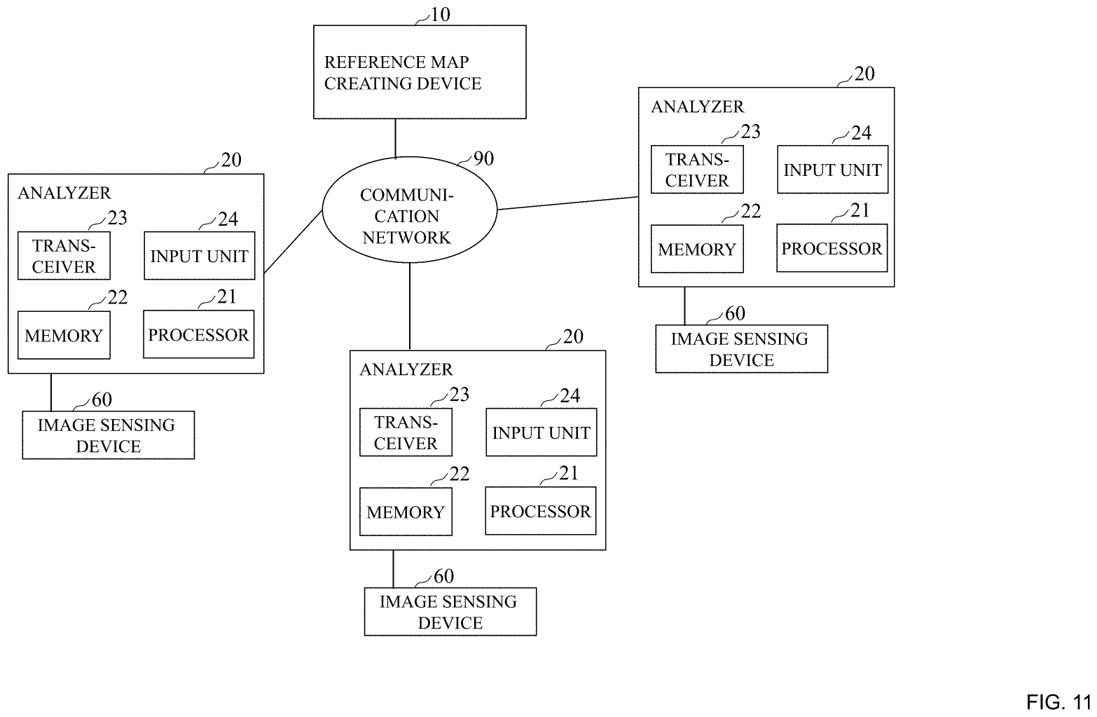

FIG. 11 illustrates an exemplary system configuration according to this embodiment. In the system according to this embodiment, each of the analyzers 20 is connected to the image sensing device 60. The image sensing device 60 is an arbitrary device capable of obtaining image data including a still image or a moving picture. For example, the image sensing device 60 includes a camera that photographs an image, a display device that displays an image, and a memory that stores image data.

The image data can be treated as spatial frequency spectral data. In this regard, according to this embodiment, the map creating unit (reference numeral 1111 in FIG. 1) provided in the reference map creating device 10 converts the image data obtained by the image sensing device 60 into vector data and creates a reference map using the vector data. For example, in the case of the image data of 30×30 pixels, the map creating unit creates vector data having nine hundreds of dimensions by setting each pixel from the image data as a dimension and setting a brightness of the pixel as a value of the corresponding dimension, and creates the reference map using the created vector data.

Here, in a case where the image data is compressed through encoding, the map creating unit decodes the image data and converts the decoded image data into vector data. If the image data contains a still image, single image data is plotted as a point on the reference map. If the image data contains a moving picture, the data can be plotted on the reference map on a frame image basis. In addition, only a part of the regions out of the image data may be extracted as the vector data. Furthermore, amplitude information of scanning lines included in a single image may be converted into vector data through frequency conversion by setting the frequency as the dimension.

Similar to other embodiments, the reference map creating device 10 transmits the reference map to each analyzer 20, and the analyzer 20 stores the reference map in the memory (reference numeral 22 in FIG. 1). In addition, as the image sensing device 60 obtains new image data, the analyzer 20 stores the new image data in the memory (reference numeral 22 in FIG. 1) as new data.

The analyzer 20 converts the new data into vector data, reads the reference map from the memory 22, and plots the new data on the reference map. In this case, in a case where the image data is a moving picture, the decoded image data are converted into the vector data. In addition, the analyzer 20 determines the attribute of the new data on the basis of the plot location. As a result, it is possible to identify the image data or determine normality/abnormality of the image.

As described above, according to this disclosure, it is possible to identify image data or determine normality/abnormality of the image in real time.

### Ninth Embodiment

As a method of plotting the new data on the reference map using the new data plotting unit 211, the method similar to that of the reference map creating unit 111 may be employed. Preferably, the method is performed without changing the reference map. In this embodiment, a specific example of the plotting method will be described below, in which the reference map does not change.

### (1) First Arrangement Example

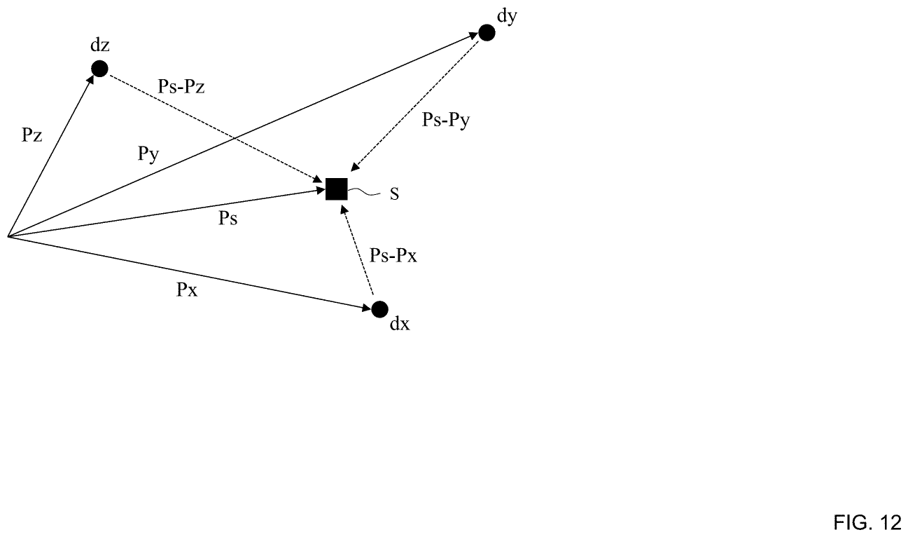

In this arrangement example, three reference data having a distance closest to the new data are selected in a multi-dimensional vector space, and the plot location of the new data is determined using the three reference data. Specifically, as illustrated in FIG. 12, distances between the new data S and each reference data in the multi-dimensional vector space are calculated, and three reference data dx, dy, and dz having the closest distance are selected. In addition, a coordinate Ps of the new data is obtained using the coordinates Px, Py, and Pz corresponding to the reference data dx, dy, and dz on the reference map. For example, the centers of the coordinates Px, Py, and Pz are set as the coordinate Ps of the new data S.

The coordinate Ps of the new data S is preferably obtained on the basis of the distances Sx, Sy, and Sz between the new data S and the reference data dx, dy, and dz in the multi-dimensional vector space. For example, a coordinate Ps satisfying the following formula is obtained. This coordinate Ps corresponds to the location of the new data S on the reference map.

(Formula 2)

|Ps−Px|:|Ps−Py|:|Ps−Pz|=Sx:Sy:Sz (2)

### (2) Second Arrangement Example

In this arrangement example, two reference data having distances closest to the new data in the multi-dimensional vector space are selected, and plot locations of the new data are determined on the basis of the two reference data. Specifically, distances between the new data and the reference data in the multi-dimensional vector space are calculated, and two reference data dx and dy having closest distances are selected similarly to FIG. 11. In addition, a coordinate Ps of the new data S is obtained using the coordinates Px and Py corresponding to the reference data dx and dy on the reference map. For example, a median between the coordinates Px and Py is selected as the coordinate Ps of the new data S.

The coordinate Ps of the new data S is preferably an internally dividing point of the coordinates Px and Py based on the distances Sx and Sy between the new data S and the reference data dx and dy in the multi-dimensional vector space. For example, the coordinate Ps that satisfies the following formula is obtained. This coordinate Ps is the location of the new data S on the reference map.

(Formula 3)

Ps=Px+(Py−Px)*Sx/(Sx+Sy) (3),

where |Ps−Px|:|Ps−Py|=Sx:Sy.

### (3) Third Arrangement Example

In this arrangement example, all the data on the reference map or “N” points having distances closest to the new data in the multi-dimensional vector space are selected, and plot locations of the new data are determined using the selected points.

Specifically, the distances between the new data and each reference data in the multi-dimensional vector space are calculated, and the “N” reference data having the closest distance are selected sequentially. In addition, the coordinates of the new data are obtained using the coordinates corresponding to the “N” reference data on the reference map. For example, a center of the coordinates of the “N” reference data is obtained. This coordinate of the center corresponds to the location of the new data S on the reference map.

In determination of the location of the new data, it is preferable to take into consideration whether or not new data is plotted in a region specified by the coordinates of a plurality of reference data having a distance close to the new data in the multi-dimensional vector space.

For example, in a case where the distance between a vector of the new data and vectors of a plurality of reference data in the multi-dimensional vector space is equal to or shorter than the distance between vectors of a plurality of the reference data in the multi-dimensional vector space, the new data is arranged in a region specified by the coordinates of a plurality of the reference data or in the vicinity of this region. In comparison, in a case where the distance between a vector of the new data and vectors of a plurality of reference data in the multi-dimensional vector space is equal to or longer than the distance between vectors of a plurality of the reference data in the multi-dimensional vector space, the new data is arranged outside of a region specified by the coordinates of a plurality of the reference data. In this manner, a relationship between the reference data and the new data can be specified on the reference map depending on whether or not the new data is plotted in a region specified by the coordinates of a plurality of the reference data having distances close to the new data in the multi-dimensional vector space.

### (4) Fourth Arrangement Example

In this arrangement example, all the data on the reference map or “N” points having distances closest to the new data in the multi-dimensional vector space are selected, and the plot location of the new data is determined by fixing a location of the data on the reference map. Specifically, data are plotted in a two-dimensional or three-dimensional space so as to maintain an actual distance between the new data and the data selected on the reference map in the multi-dimensional space to the maximum. As a method thereof, various methods are known, such as a Principal Component Analysis method, a t-Distributed Stochastic Neighbor Embedding (t-SNE) method and a Uniform Manifold Approximation and Projection for Dimension Reduction (UMAP) method. In this method, in a case where the new data is significantly different from the data on the reference map, its plot location is outside a plot range of the data on the reference map.

As described above, the plot location of the new data is preferably determined using a part or all of the reference data. Here, the distance in the multi-dimensional vector space may be an Euclidean distance or an inner product space distance or may be obtained using an arbitrary computation method such as a method of using an outer product.

This disclosure is applicable to an information communication industry.

### REFERENCE SIGNS LIST

10: reference map creating device
20: analyzer
11, 21: processor
12, 22: memory
13, 23: transceiver
111: reference map creating unit
1111: map creating unit
1112: attribute defining unit
211: new data plotting unit
212: attribute determination unit
213: additional area defining unit
30: vehicle
31, 32: sensor
40A, 40B, 40C: computer
50: telephone set
60: image sensing device
90: communication network

What is claimed is:

1. An analyzer comprising:
a reference map receiver configured to receive a reference map in which reference data are plotted in advance on a map on the basis of similarity index between the reference data; and
a new data plotting unit configured to plot new data different from the reference data on the reference map on the basis of similarity index between the new data and all or a part of the reference data on the reference map.

2. The analyzer according to claim 1, wherein a plurality of areas are defined on the reference map,
an area attribute representing an attribute unique to the area is defined for each area, and
the analyzer further comprises a new data attribute determination unit configured to determine an attribute of the new data on the basis of which of the areas a plot location of the new data belongs to.

3. The analyzer according to claim 2, wherein the reference data and the new data are sensor data detected from a sensor mounted on a device or data obtained from a device or a component provided in the device, and
the area attribute includes a state of the device or the component.

4. The analyzer according to claim 2, wherein the reference data and the new data are vibration data detected using a sensor mounted on a vehicle, and
the area attribute includes a road surface state where the vehicle travels.

5. The analyzer according to claim 2, wherein the reference data and the new data are biological data detected from a living body, and
the area attribute includes a state of the living body.

6. The analyzer according to claim 2, wherein the reference data and the new data are sound frequency spectral data, and
the area attribute includes a talker's psychological state.

7. The analyzer according to claim 2, wherein the reference data and the new data are image data, and
the area attribute includes identification information of the image data.

8. The analyzer according to claim 2, further comprising an additional area defining unit configured to define a new area including the new data and an area attribute of the area if the new data does not belong to any one of the areas.

9. An analysis system comprising:
the analyzer according to claim 1; and
a reference map creating device provided with a reference map creating unit that obtains data used as the reference data and creates the reference map on the basis of similarity index between the reference data and configured to provide the reference map to the analyzer.

10. An analysis method, wherein an analyzer, comprising:
receiving a reference map created in advance on the basis of similarity index between reference data; and
plotting new data different from the reference data on the reference map on the basis of similarity index between the new data and all or a part of the reference data on the reference map.

11. The analysis system according to claim 9, wherein a plurality of areas are defined on the reference map,
an area attribute representing an attribute unique to the area is defined for each area, and
the analyzer further comprises a new data attribute determination unit configured to determine an attribute of the new data on the basis of which of the areas a plot location of the new data belongs to.
